(19) United States
(12) Patent Application Publication
(10) Pub. No.: US 2021/0401738 A1
(43) Pub. Date: Dec. 30, 2021

(54) DIETARY SUPPLEMENT COMPOSITIONS

(71) Applicant: AGRONOMED PHARMACEUTICALS LLC, Exton, PA (US)

(72) Inventor: Jonathan COHN, Exton, PA (US)

(73) Assignee: AGRONOMED PHARMACEUTICALS LLC, Exton, PA (US)

(21) Appl. No.: 17/293,272

(22) PCT Filed: Nov. 14, 2019
(86) PCT No.: PCT/US19/61515

§ 371 Date: May 12, 2021

Related U.S. Application Data
(60) Provisional application No. 62/767,487, filed on Nov. 14, 2018.

(51) Int. Cl. A61K 9/00; A61K 36/185; A61K 36/9068; A61K 31/05; A61K 31/10; A61K 33/38; A61K 31/23; A23L 33/105; A23L 33/115; A23L 33/16; A23K 50/40; A23K 10/30; A23K 20/158; A23K 20/20

(52) U.S. Cl. CPC A61K 9/0056; A23V 2002/00; A61K 36/9068; A61K 31/05; A61K 31/10; A61K 33/38; A61K 31/23; A23L 33/105; A23L 33/115; A23L 33/16; A23K 50/40; A23K 10/30; A23K 20/158; A23K 20/20; A61K 36/185

(57) ABSTRACT

A cannabinoid (CBD) and methylsulfonylmethane (MSM) formulation for ameliorating the symptoms of one or more of Autoimmune diseases, muscle and joint inflammation and pain, and psychological disorders such as; anxiety, fear, and panic in canines, The method of administration is an oral chewable supplement. Other ingredients within the chewable will help with internal dissemination of the CBD and MSM, and give added nutrients to the canine in a pleasant formulation.

### PRIORITY

This application claims the benefit under 35 U.S.C. § 119(e) to U.S. Provisional Application Ser. No. 62/767,487 filed on Nov. 14, 2018, the entirety of which is incorporated herein by reference.

### FIELD OF THE INVENTION

The present invention is generally directed to a dietary supplement composition, and more particularly a chewable composition that includes one or more of each of cannabinoids, organosulfur compounds, medium-chain triglycerides, and colloidal silver in a suitable edible carrier.

### BACKGROUND OF THE INVENTION

Due to the playful, energetic, and even hard-working nature of domestic animals, specifically dogs, joint and muscle conditions are often very common. Throughout a pet's life, the pet owners want them to live the best quality of life available. Unfortunately, some pets develop autoimmune diseases that owners would like to help ease or minimize the symptoms of. Dogs suffering from the symptoms of these diseases and conditions are often uncomfortable and in severe pain.

Pets may have behavioral conditions as result of a plethora of causes. Dogs suffering from anxiety or fear can become aggressive with people and other animals. Depending on the severity of aggression, owners can face litigation and the dog can be euthanized. Dogs may suffer from separation anxiety which may lead to them destroying the owner's house or belongings. This is stressful to the owner but may also stress the dog when disappointing their owner or being punished. Mild fears over loud noises or strange unknowns to the dog may cause the dog to bark constantly or even hide. The constant barking can be an annoyance to the owner, and the noises cause unnecessary stress to the dog. Even hyperactive or overly excitable dogs cause problems for owners and themselves. The constant running, barking, and seeking attention can irritate owners, and lead to them giving the dog away.

Currently pet owners must make difficult decisions to end their beloved dog's life due to pain from conditions and diseases that their dog is suffering from. While there are current medications that help with treatment of diseases, they will not increase the quality of life for the dogs. Surgeries to help a dog may be too expensive for the owners. Steroids, harsh pain medication, and supplements may not work at all or may decrease in effectiveness over time. These issues lead to premature euthanasia decisions for pet owners.

Harsh pain killers and anti-inflammatory drugs called Non-steroidal Anti-Inflammatory Drugs (NSAID) can have severe effects on the dogs. While they may stop the pain, or help minimize the inflammation they can cause the dog to have reactions such as: not eating or eating less, lethargy, depression, changes in behavior, vomiting, diarrhea, black tarry-colored stool, yellowing of gums, skin, or the whites of the eyes, change in drinking, and changes in skin such as scabs, redness, or scratching. Most of these symptoms are poor quality of life for dogs.

### SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

The compositions hereof are characterized, in various embodiments, as comprising one or more of each of cannabinoids, organosulfur compounds, medium-chain triglycerides, and colloidal silver in a suitable edible carrier.

In an exemplary embodiment, a dietary supplement composition including one or more cannabinoids, one or more organosulfur compounds, one or more medium-chain triglycerides, colloidal silver, and an edible carrier.

In an exemplary embodiment, a method of administering a cannabinoid to a canine includes orally administering a cannabinoid composition to a canine. The cannabinoid composition comprises includes one or more cannabinoids, one or more organosulfur compounds, one or more medium-chain triglycerides, colloidal silver, and an edible carrier.

In an exemplary embodiment, dietary supplement composition includes cannabidiol (CBD), the organosulfur compound methylsulfonylmethane (MSM), one or more medium-chain triglycerides (MCTs), and colloidal silver in a suitable edible carrier. According to the exemplary embodiment, the composition is provided in an edible biscuit form. In some embodiments, the compositions include ginger. In certain embodiments, the edible biscuit is formulated as a pet, or more particularly, a dog biscuit, and comprises in addition to the foregoing listed components peanut butter, flour, baking powder, and lactose free milk. In some particular embodiments, the compositions may further include one or more flavorings, proteins selected from animal and vegetable derived proteins, and any one or more fruits, vegetables and meats and combinations of these. In some examples, the compositions include one or more of blueberry, pumpkin, sweet potato, turkey, fish, venison, lamb and beef.

Other features and advantages of the present invention will be apparent from the following more detailed description, taken in conjunction with the accompanying drawings which illustrate, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 shows a CBD and MSM enriched oral chewable animal supplement, according to an embodiment.

FIG. 2 shows a flowchart illustrating a method of making a cannabinoid and MSM enriched oral chewable dog supplement, according to an embodiment.

FIG. 3 shows data related to a study with the supplement, identified as Appendix Table 1: Study Cohort.

FIG. 4 shows data related to a study with the supplement identified as Appendix Table 2: Study Cohort Intake Data.

FIG. 5 shows data related to a study with the supplement, identified as Appendix Table 3: Study Cohort Intake Data Continued.

FIG. 6 shows data related to a study with the supplement, identified as Appendix Table 4: Study Cohort Intake Data Continued.

FIG. 7 shows data related to a study with the supplement, identified as Appendix Table 5: Study Cohort Conclusion Data.

FIG. 8 shows data related to a study with the supplement, identified as Appendix Table 6: Study Cohort Conclusion Data Continued.

FIG. 9 shows data related to a study with the supplement, identified as Appendix Table 7: Study Cohort Summary Symptom Data (by dog, by date).

Wherever possible, the same reference numbers will be used throughout the drawings to represent the same parts.

### DETAILED DESCRIPTION OF THE INVENTION

The present invention combines CBD and MSM in an oral chewable drug in the form of a treat/biscuit. The composition provides a positive result for autoimmune stimulation, anxiety relief, as well as pain and inflammation relief on bone and joint pain. The invention was originally designed to provide relief for pain and anxiety, the unexpected result was an autoimmune stimulation. The composition also provides the unexpected feature of extended shelf stability as compared to other edible compositions, the stability owing to the combination of at least the MCT component, and in particular in the presence of ginger.

Cannabis is becoming more widely used within human medicine. It is yet to be recognized as a good option in veterinary medicine. Cannabidiol (CBD) is a cannabinoid with high therapeutic potential without psychoactive effects; unlike Tetrahydrocannabinol (THC), the psychoactive lipid in the cannabis plant. THC has high toxicity potential in animals due to their heightened Endocannabinoid System, which absorbs the lipid causing neurological toxicity. CBD does not cause any psychoactive effects and is not known to be toxic to canines.

The present invention is an oral, chewable drug that uses the combination of cannabidiol (CBD) and Methylsulfonylmethane (MSM) to ameliorate (one or more of help prevent, treat, delay, and ease) the symptoms of one or more of symptoms in dogs with autoimmune diseases, muscle and joint conditions causing inflammation and pain, and psychological behaviors from anxiety and fear.

Without being bound by theory, the inventors have formulated the compositions with the aim that the CBD will interact with the animal's Endocannabinoid System (ES), which binds to Cannabinoid receptors within the animal's body to help maintain physiological homeostasis. It is further believed that MSM will affect the flexibility and permeability of cell walls to enable fluids to pass through cell tissues more easily.

The foregoing ingredients work in concert with bio-active Silver Hydrosol to help promote the healing of internal illnesses and discomfort. The combination of the three agents together are designed to interact with the animal's body to help manage and ameliorate the symptoms of autoimmune diseases, muscle and joint inflammation and pain, and psychological conditions, including anxiety.

It is further believed that CBD, MSM, and Silver Hydrosol synergistically interact to assist the body in achieving homeostasis. It is believed by the inventors that CBD attaches to the Endocannabinoid System's receptors, MSM aids in the absorption of the CBD, and Silver Hydrosol by the cells thus benefiting the healing process.

Cannabinoids

In the various embodiments, the dietary supplement includes one or more cannabinoid compounds. According to the various embodiments, the cannabinoids may include any extract, such as an oil from any of cannabis plants. CBD, for example, is obtained from Industrial Hemp plants via extraction using CO₂ and/or ethanol extraction processes. Generally, cannabinoid means and refers to one cannabinoid or a mixture of cannabinoids, cannabinoid isolates, or any other isolated and purified cannabinoid, terpene, or biomolecule product by the genus Cannabis plant, for example, cannabidiol (CBD). Cannabidiol (CBD), cannabigerol (CBG), cannabinol (CBN), cannabichromene (CBC), and other cannabinoids are sourced from industrial hemp plants with THC levels below 0.3 weight percent. Cannabinoids are extracted from the flower material using ethanol extraction and/or CO₂ extraction methods. Cannabinoids may then be refined and further processed to an isolate having high purity. The cannabinoid isolate(s) used are approximately 98-99% pure.

In accordance with the various embodiments, the amount of one or more cannabinoids present in the composition can range from about 0.5 weight percent to about 3 weight percent, and in some embodiments from about 0.5 weight percent to about 2.5 weight percent, and in some embodiments from about 1 weight percent to about 3 weight percent or any suitable combination, sub-combination, range, or sub-range thereof by weight, based on the weight of the composition. In some embodiments according to the disclosure, the composition includes at least about 0.5% of the one or more cannabinoid. Thus, in some embodiments, the at least one cannabinoid is present in an amount that is not less than about 0.5%, based upon the total weight of the composition. In some embodiments, two or more cannabinoids may be present.

Thus, one or a combination of cannabinoids may be present, by weight, based on the total weight of the composition, from about 0.5, 0.6, 0.7, 0.8, 0.9, 1.0, 1.5, 2.0, to about 2.5 weight percent, including increments and ranges therein and there between.

Medium-Chain Triglycerides (MCTs)

In the various embodiments, the dietary supplement includes one or more medium-chain triglycerides compounds. Medium-chain triglycerides (MCTs) are triglycerides with two or three fatty acids and an aliphatic tail of from 6 to 12 carbon atoms (medium-chain fatty acids). Sources for commercial extraction of MCTs include palm kernel oil and coconut oil. The lipids in MCTs include caproic acid (C6, also known as hexanoic acid), caprylic acid (C8, octanoic acid), capric acid (C10, decanoic acid), and lauric acid (C12, dodecanoic acid).

In accordance with the various embodiments, the amount of one or more MCTs present in the composition can range from about 2.5 to about 5.0% or any suitable combination, sub-combination, range, or sub-range thereof by weight, based on the weight of the composition. In some embodiments, the composition includes at least about 2.5% of the one or more MCT. Thus, in some embodiments, the one or more MCT is present in an amount that is not less than about 2.5%, based upon the total weight of the composition. In some embodiments, two or more MCTs are present.

Thus, one or a combination of MCTs may be present, by weight, based on the total weight of the composition, from about 2.5, 3.0, 3.5, 4.0, 4.5 to about 5.0 weight percent, including increments and ranges therein and there between.

Colloidal Silver

In the various embodiments, the dietary supplement includes one or more silver containing compounds. Silver hydrosol, also known as colloidal silver, is silver particles suspended in a liquid. In accordance with the various embodiments, the amount of silver hydrosol present in the composition may result in dosages of less than about 5 micrograms per kilogram (μg/kg) per day.

Organosulfer Compounds

In the various embodiments, the dietary supplement includes one or more organosulfer compounds. Methylsulfonylmethane (MSM) is an organosulfur compound with the formula (CH₃)₂SO₂. It is closely related to dimethyl sulfoxide (DMSO). Raw material source of MSM is provided at a concentration of 100%.

In accordance with the various embodiments, the amount of one or more organosulfur compounds present in the composition can range from about 0.75 weight percent to about 2 weight percent or any suitable combination, sub-combination, range, or sub-range thereof by weight, based on the weight of the composition. In some embodiments according to the disclosure, the composition includes at least about 0.75 weight percent of the one or more organosulfur compound. Thus, in some embodiments, the at least one organosulfur compound is present in an amount that is not less than about 0.75 weight percent, based upon the total weight of the composition. In some embodiments, two or more organosulfur compounds are present.

Thus, one or a combination of organosulfur compounds may be present, by weight, based on the total weight of the composition, may be about 0.75, about 1.0, about 1.25, about 1.5, and/or about 1.75, to about 2.0 weight percent, including increments and ranges therein and there between. In some embodiments, the organosulfur compounds may be present, per dose, of about 75 milligrams to about 100 milligrams.

Ginger

In the various embodiments, the dietary supplement includes ginger. Ginger is a plant with leafy stems and yellow-ish green flowers. Ginger root extract is commonly used for relief of various types of stomach problems. Ginger contains various active ingredients, such as gingerenone A, zingerone, shogoals, paradol, gingerol, and 1-dehydro-10-gingerdione, that may reduce nausea and inflammation. Ginger has also been used for centuries as a natural food preservative.

In accordance with the various embodiments, one or a combination of ginger compounds may be present. In some embodiments, the total weight of ginger compounds by weight, based on the total weight of the composition, may be about 0.5, about 1.0, and/or about 1.25, to about 1.5 weight percent based on the total weight of the composition, including increments and ranges therein and there between.

Other Ingredients

In the various embodiments, the dietary supplement includes one or more additional ingredients, which may include milk or lactose free milk, peanut butter, flour, baking powder, oils and other food-based ingredients, flavorings and actives. Lactose-free milk is generally known as milk-based product that includes milk (reduced fat, low-fat, or fat-free depending or other forms), lactase enzyme, Vitamin A, and Vitamin D. In alternate embodiments, the composition may comprise alternate milk forms, including lactose containing forms. Yet other components that may be added to the compositions are selected from any suitable edible material, including but not limited to one or more flavorings, proteins selected from animal and vegetable derived proteins, and any one or more fruits, vegetables and meats and combinations of these. In some examples, the compositions include one or more of blueberry, pumpkin, sweet potato, turkey, fish, venison, lamb and beef.

### Example Composition

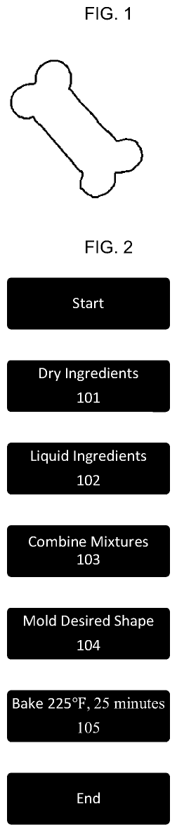

The cannabinoid enriched oral chewable drug (FIG. 1) is comprised of CBD isolate, MSM, Silver Hydrosol (colloidal silver), Organic Peanut Butter, Flour, Baking Powder, Ginger, MCT oil, and lactose free milk (such as Lactaid™ Milk).

In the example of FIG. 2, a cannabinoid enriched oral chewable composition is prepared. The cannabinoid enriched oral chewable composition may be the same or different from the cannabinoid enriched oral chewable composition of FIG. 1. At block 101, the dry ingredients are mixed in a first bowl or mixer. At block 102, the liquid ingredients are mixed in a second bowl or mixer. At block 103, the dry ingredients and the liquid ingredients are combined and blended to a substantially uniform composition. At block 104, the combined mixtures are molded into a desired shape. At block 105, the combined mixture is baked to produce the cannabinoid enriched oral chewable composition. In some embodiments, the combined mixture may be baked at a temperature of about 225 degrees Fahrenheit for a time of about 25 minutes.

An example cannabinoid enriched oral chewable composition which may be prepared by the method of FIG. 2 is shown below in Table 1.

TABLE 1

| Dry Ingredients | Liquid Ingredients (Final vol 1 cup) |
|---|---|
| 128 mg CBD isolate | 15 drops of Silver Hydrosol |
| 9 grams MSM | 4 tbsp. MCT oil |
| 2 cups of Whole meal Flour | 270 g. Organic peanut butter |
| 1 tbsp. Baking powder | Balance of cup Lactaid Milk |
| 1 tsp. Ginger | |

The materials of Table 1 were prepared as a cannabinoid enriched oral chewable composition by the following steps. The dry ingredients were combined and mixed together to ensure even distribution of CBD and MSM. Organic peanut butter was then added to the dry mixture. The MCT palm oil, Lactaid Milk, and Silver Hydrosol were combined in a separate bowl to form a liquid mixture. The liquid mixture was poured into the bowl with dry mixture and organic peanut butter and mixed together. The resulting dough was rolled out to a desired thickness for molding. The composition was molded and checked for correct weight, about 6 to about 9 grams per chewable supplement. An oven was preheated to 225 degrees Fahrenheit and the molded composition was baked on trays for 25 minutes and cooled thoroughly.

Other flavors can be added to the cannabinoid enriched oral chewable supplement such as using chicken or lamb flavoring or even fruit or vegetable flavors to provide variety in the dog's diet. Changing the amount CBD, MSM, or Silver Hydrosol can change the makeup of FIG. 1. It is believed that the CBD, MSM, and Silver Hydrosol work in conjunction with the animal's Endocannabinoid System and cellular structure by being absorbed in the small intestine through the continued enzyme break down of food and absorption by villi on the lining of intestinal wall.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

While the invention has been described with reference to one or more embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

### Examples: Study

The cannabinoid enriched oral chewable composition was then feed to various canines exhibiting various symptoms. The number of treats and thus the dosage of cannabinoids are based on weight of the dog, approximately one treat per 20 lbs. is the recommended dose. In some embodiments, the CBD is present at a dosage of about 0.66 milligrams per 20 pounds (0.03 mg/lb) (0.073 mg/kg). In some embodiments, the dosage may be increased or decreased by about 0.05 milligrams (mg) per treat. In some embodiments, the dosage may vary between about 0.067 mg/kg and 0.078 mg/kg. The frequency can be determined by the severity of disease, disorder, or condition of the dog.

### Examples: Study Data Appendix

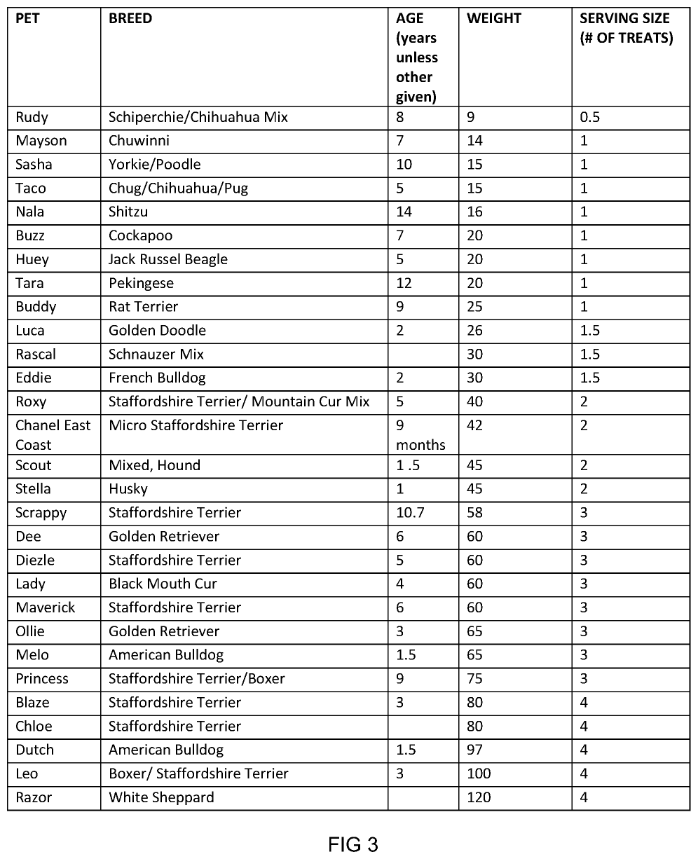

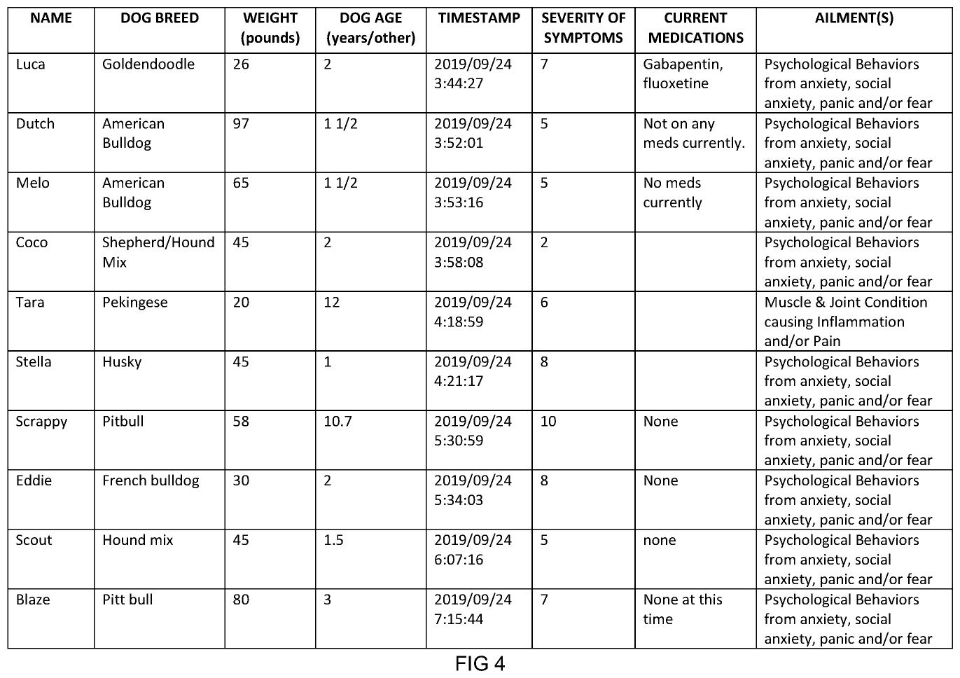

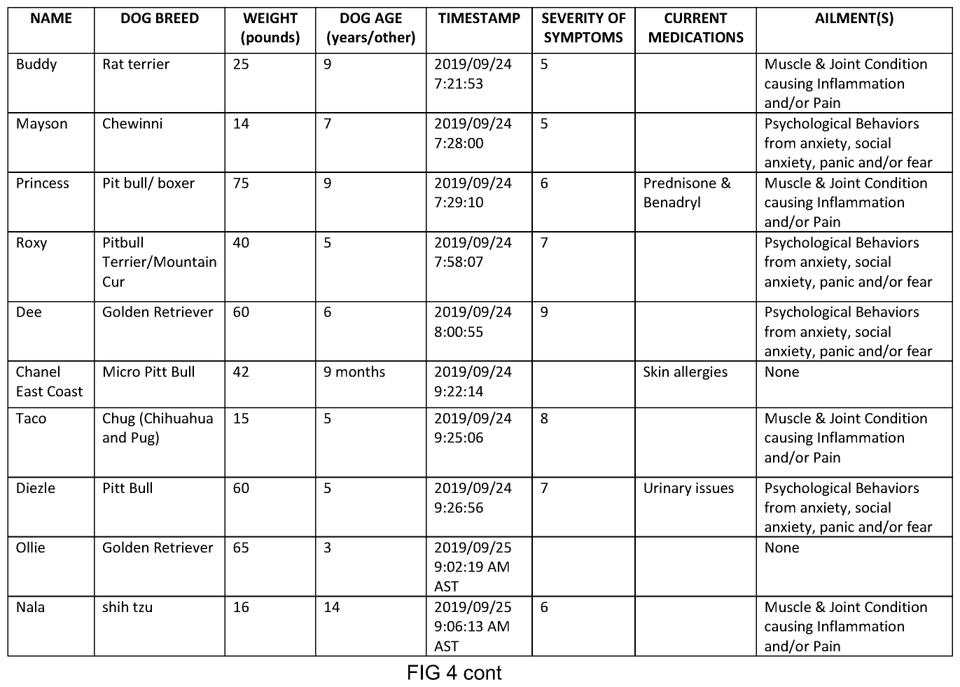

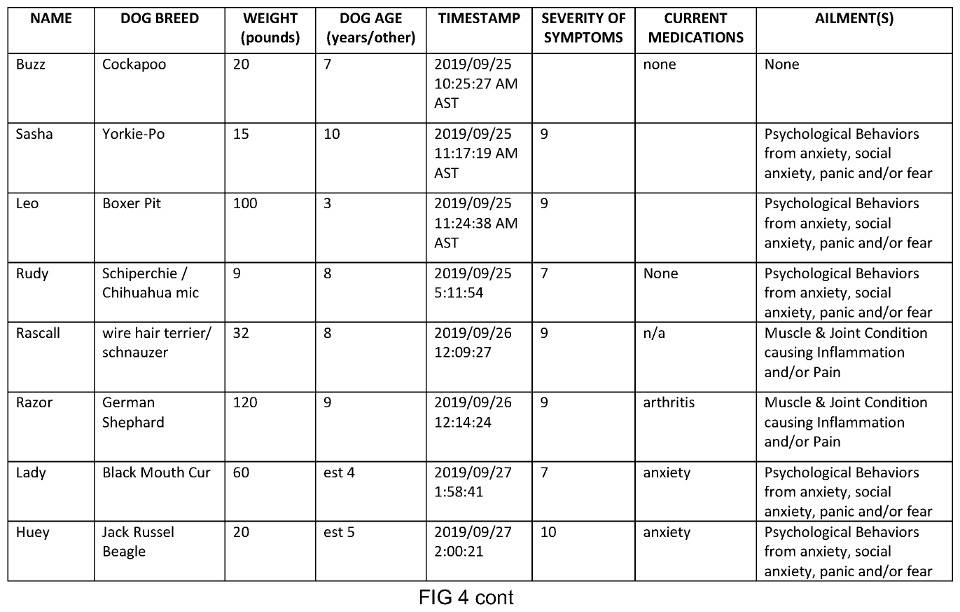

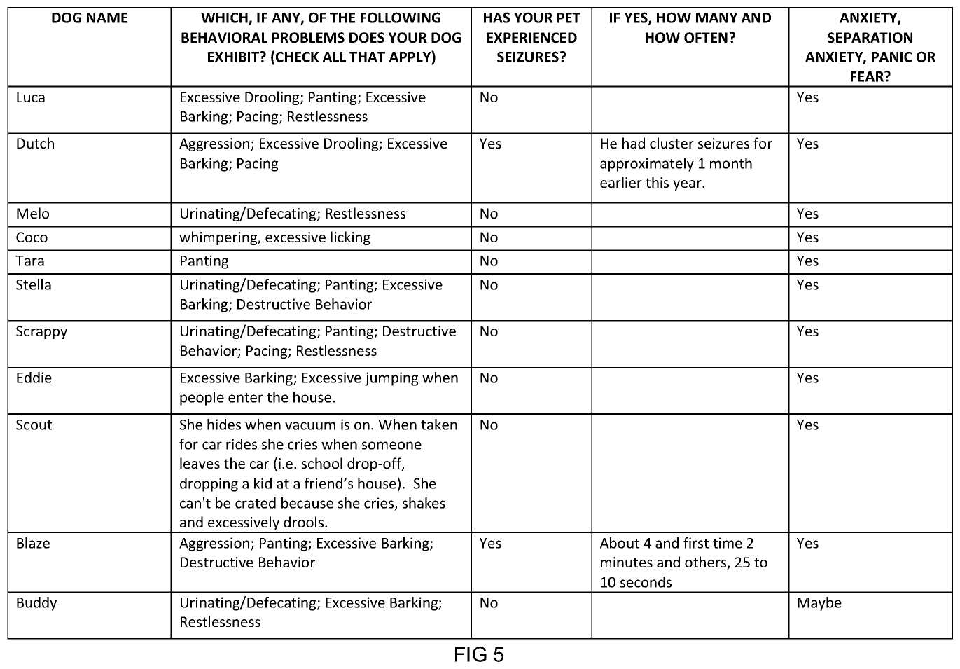

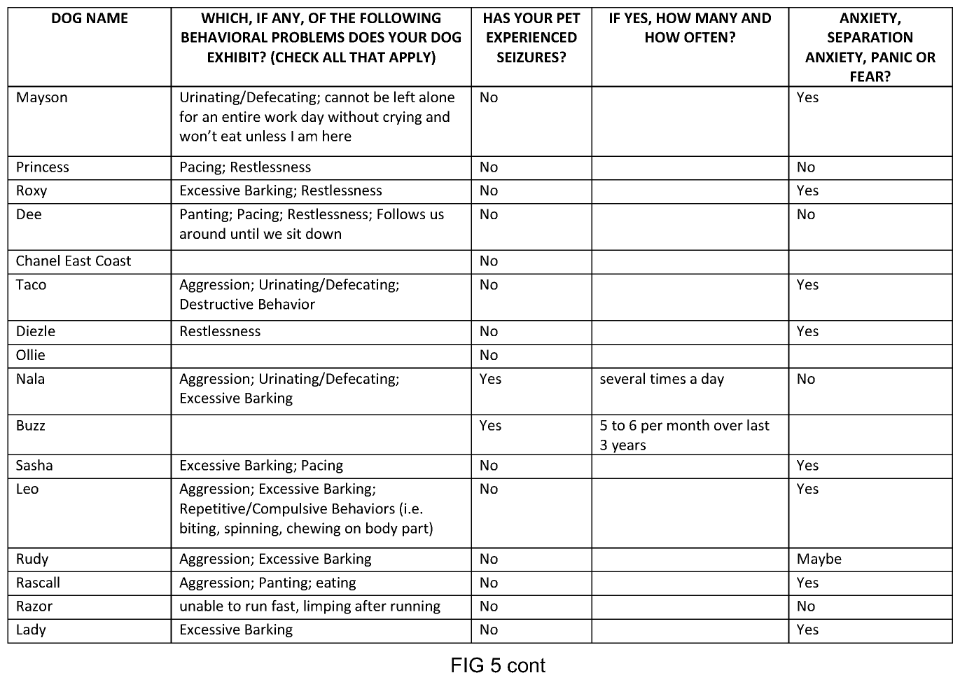

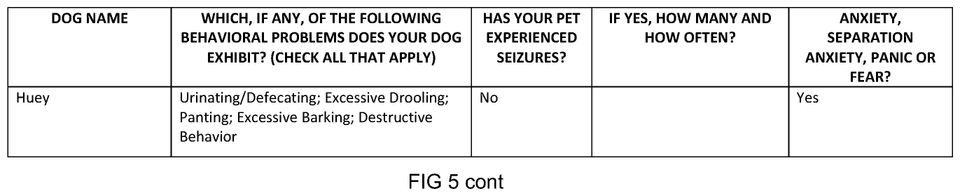

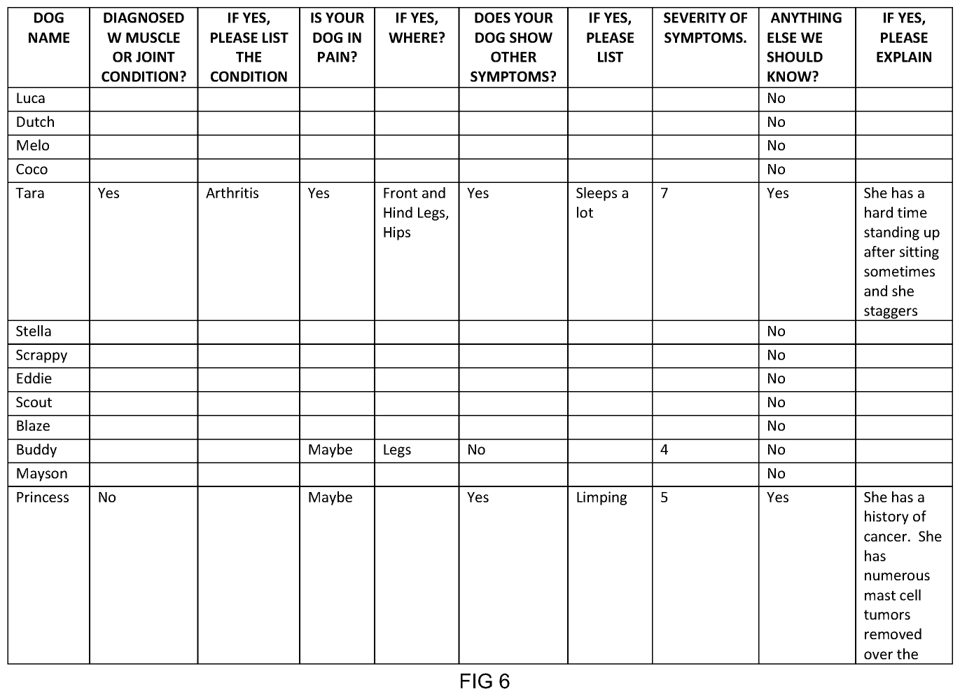

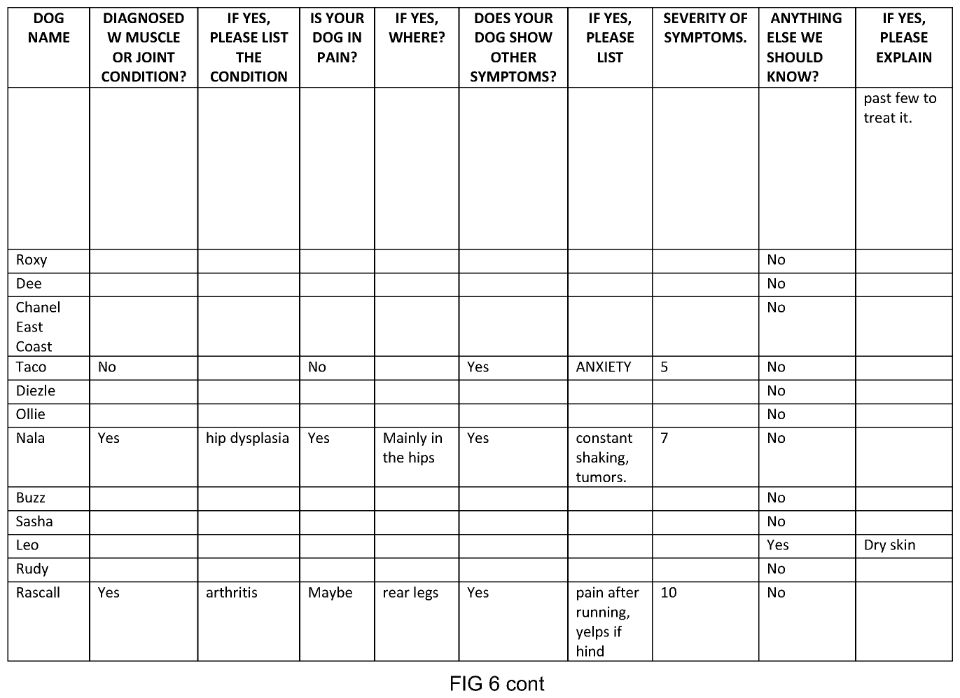

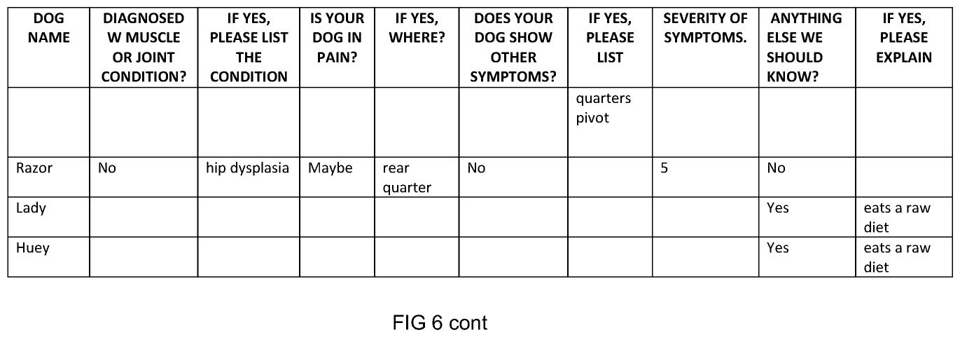

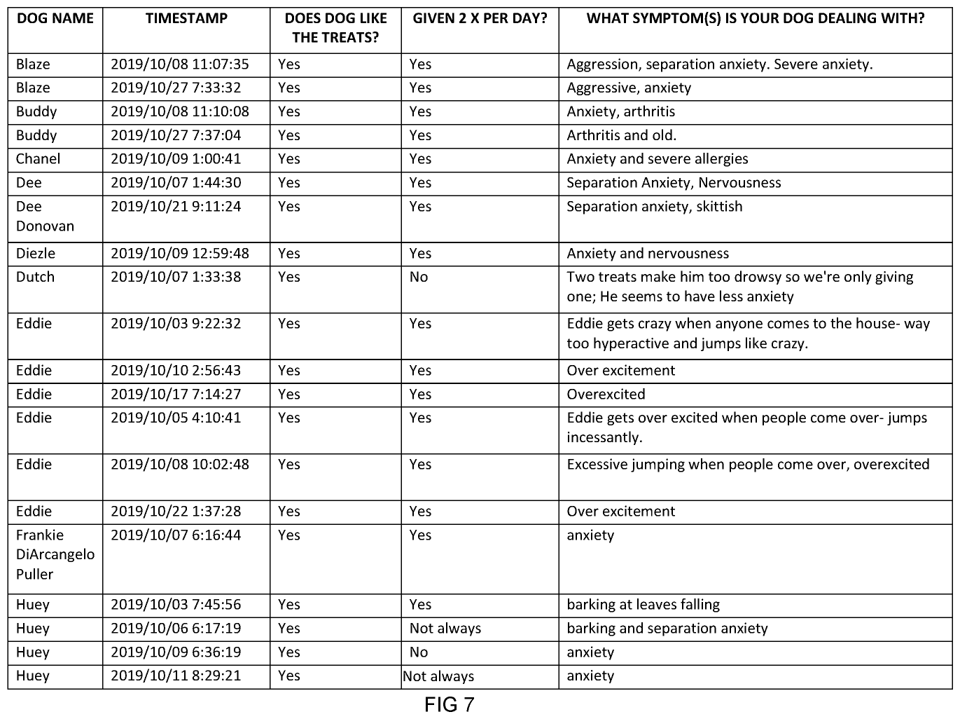

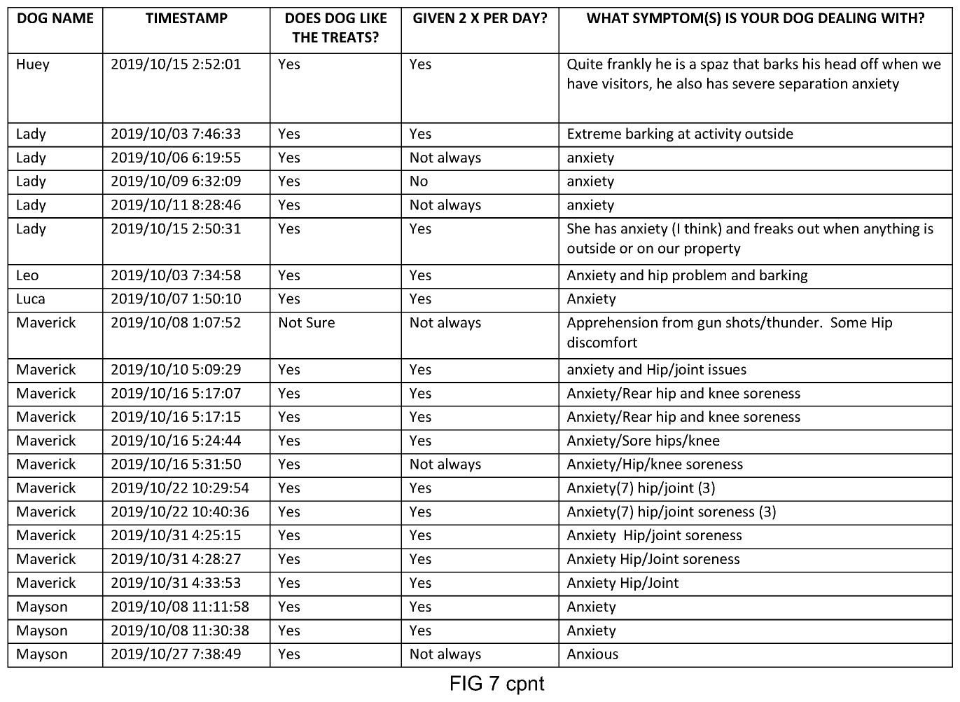

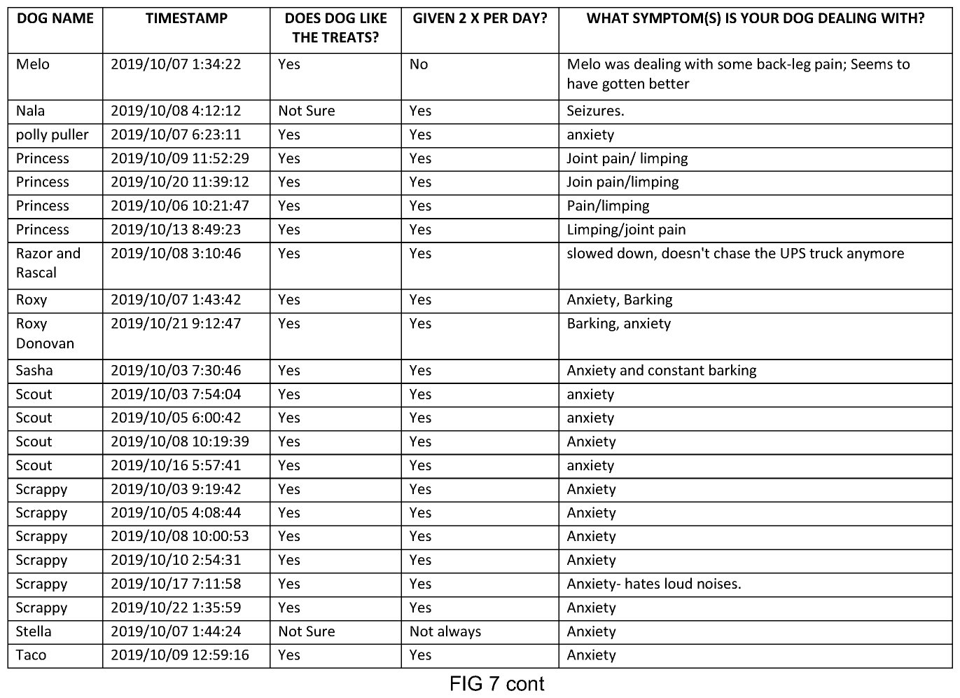

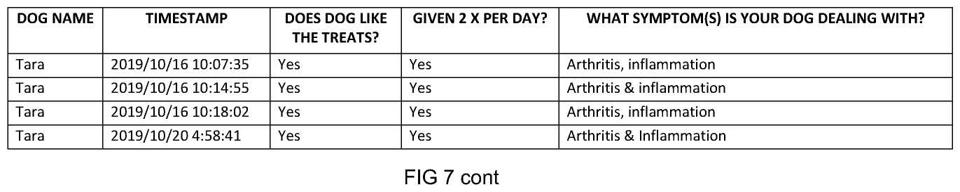

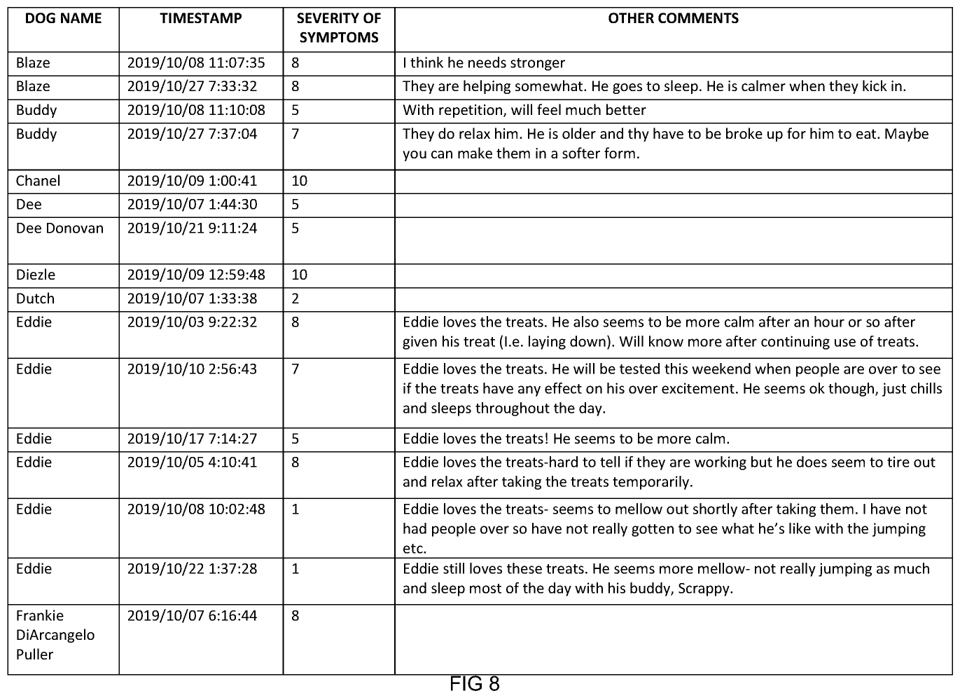

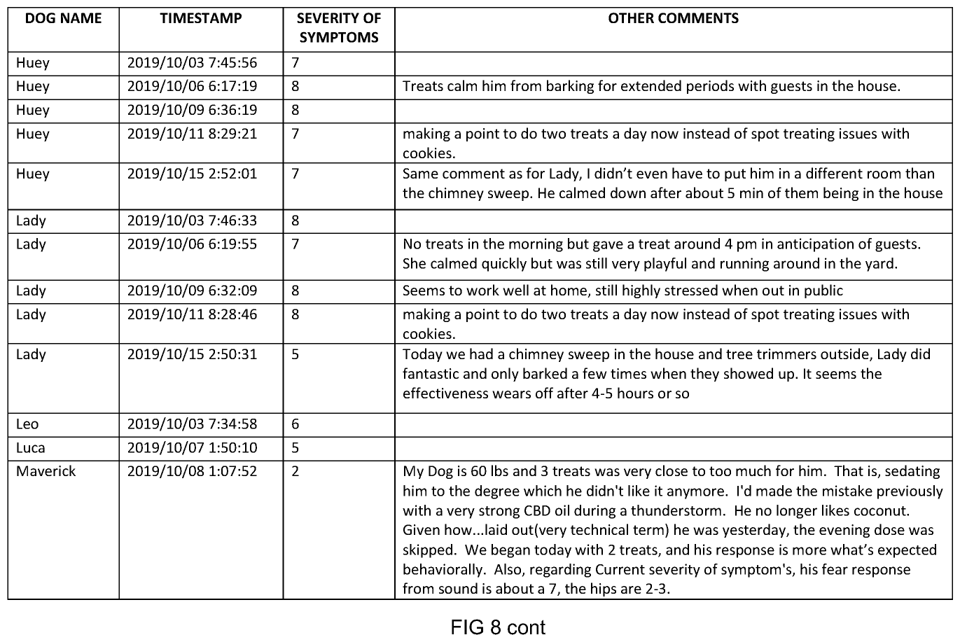

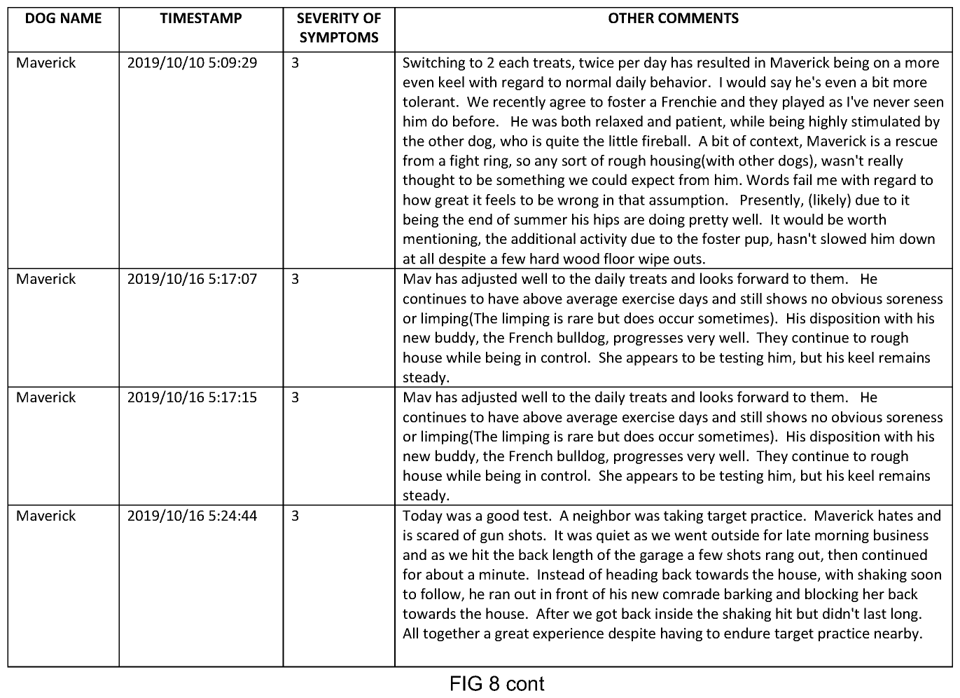

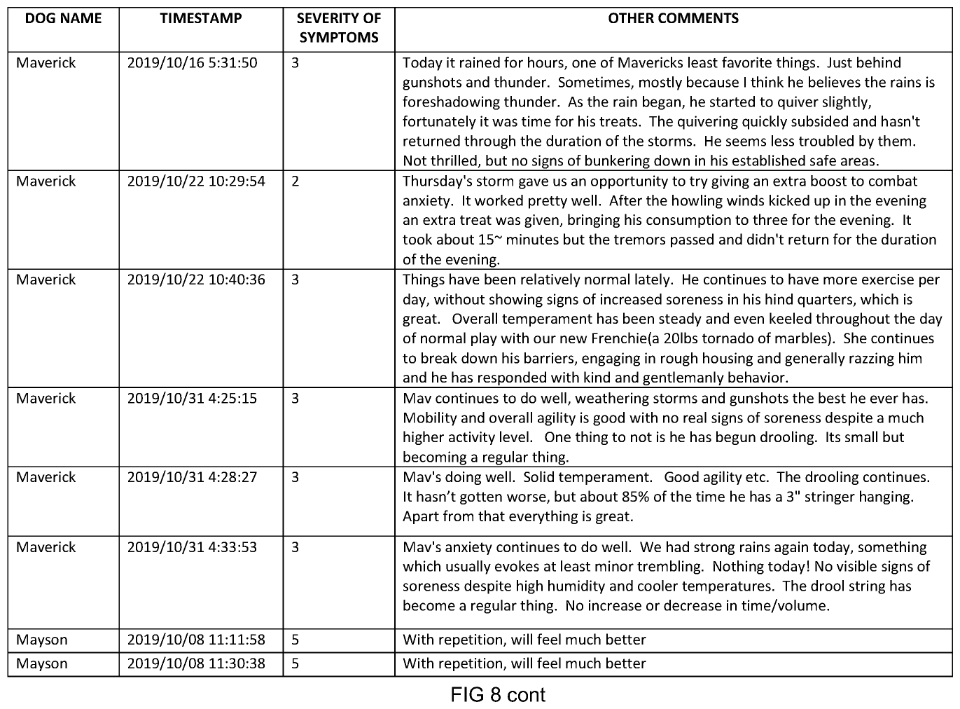

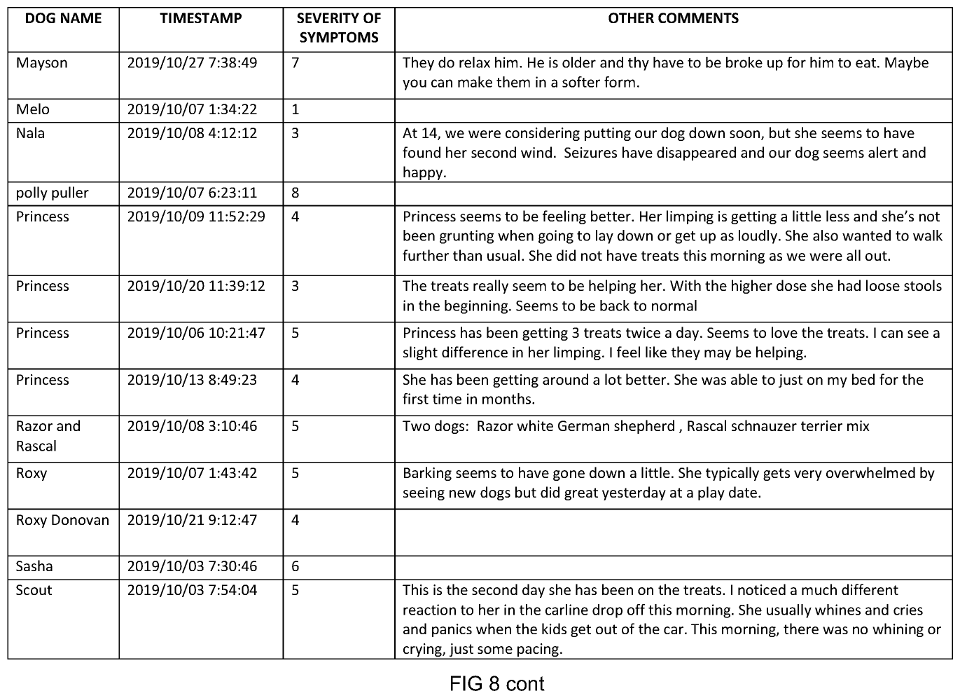

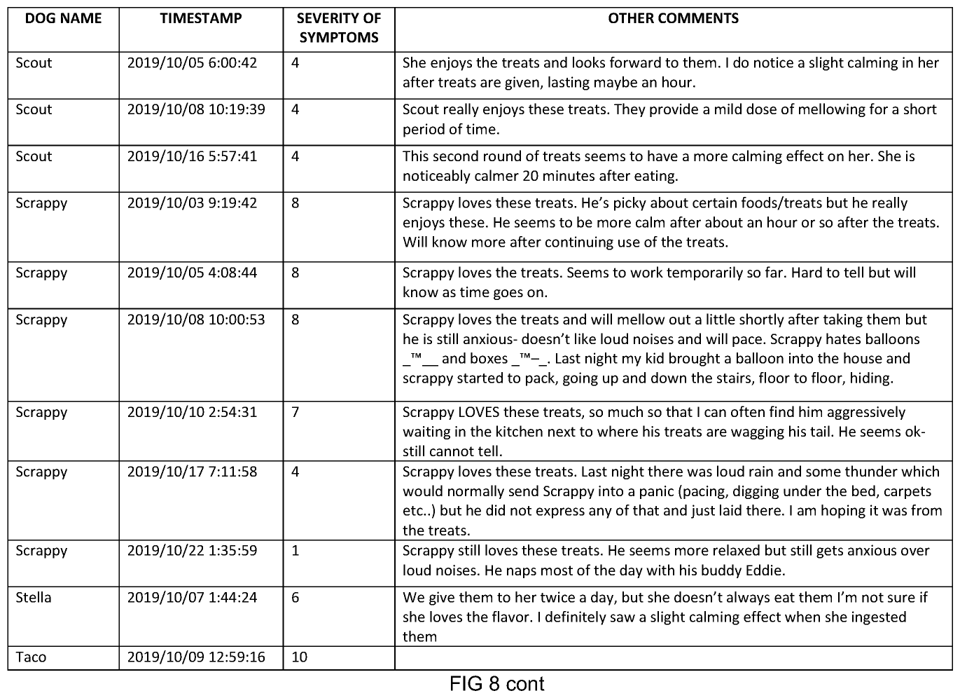

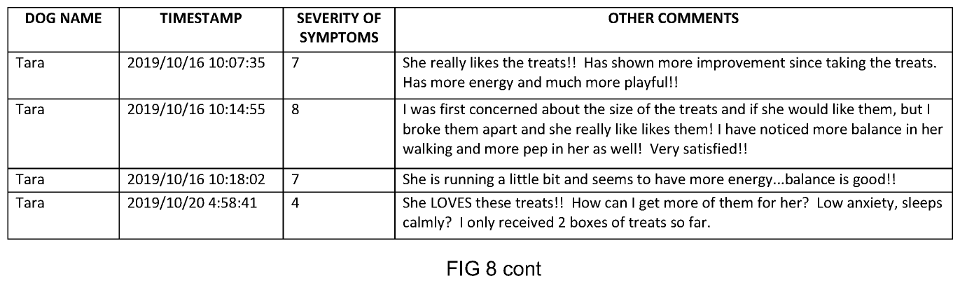

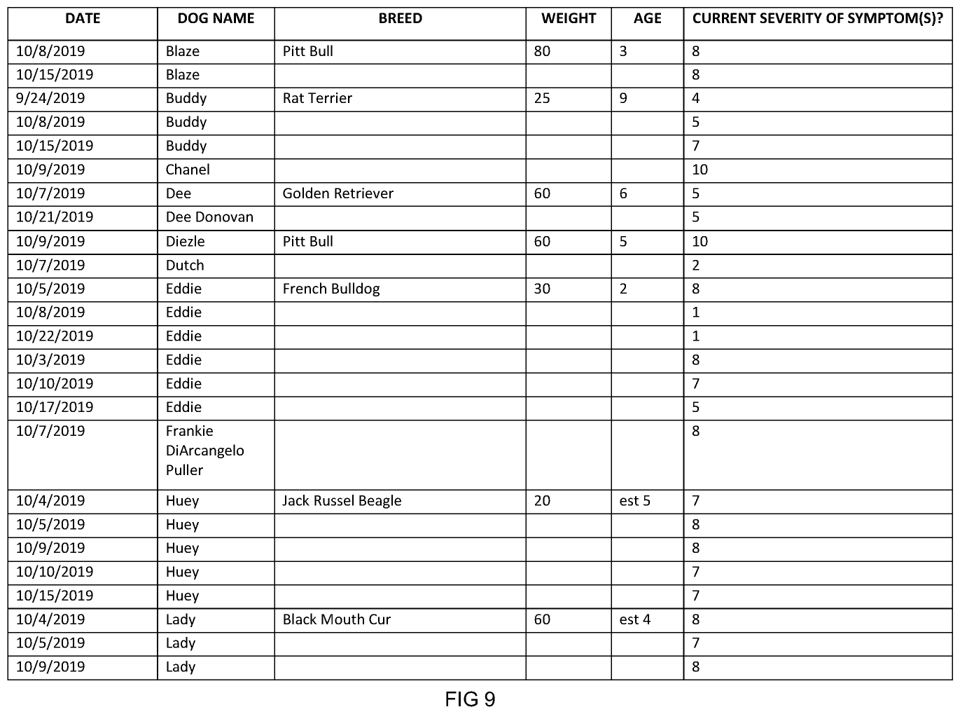

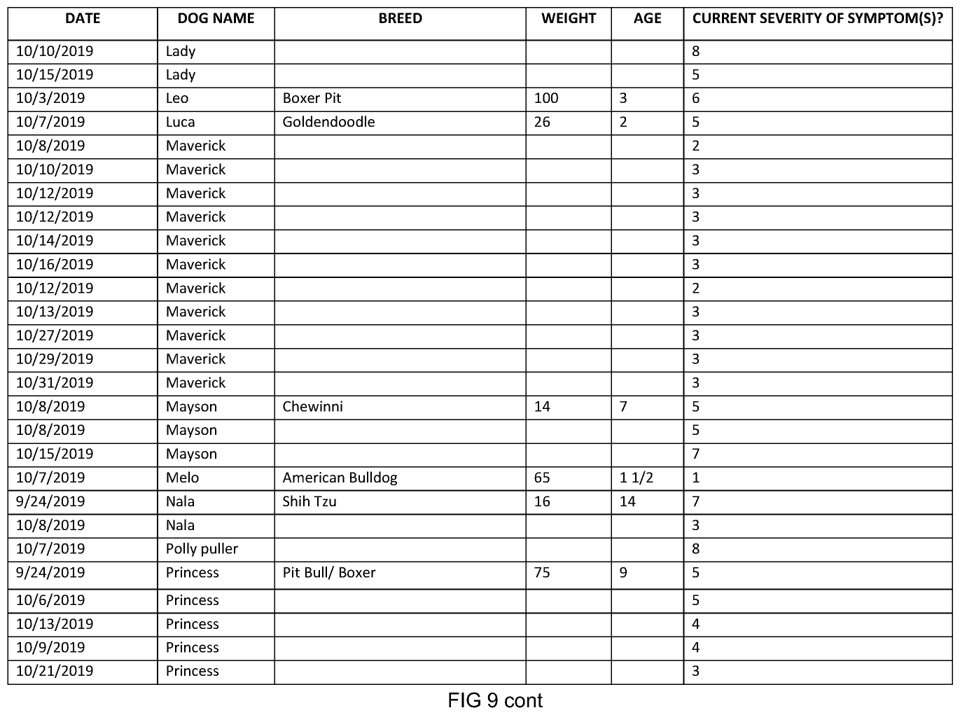

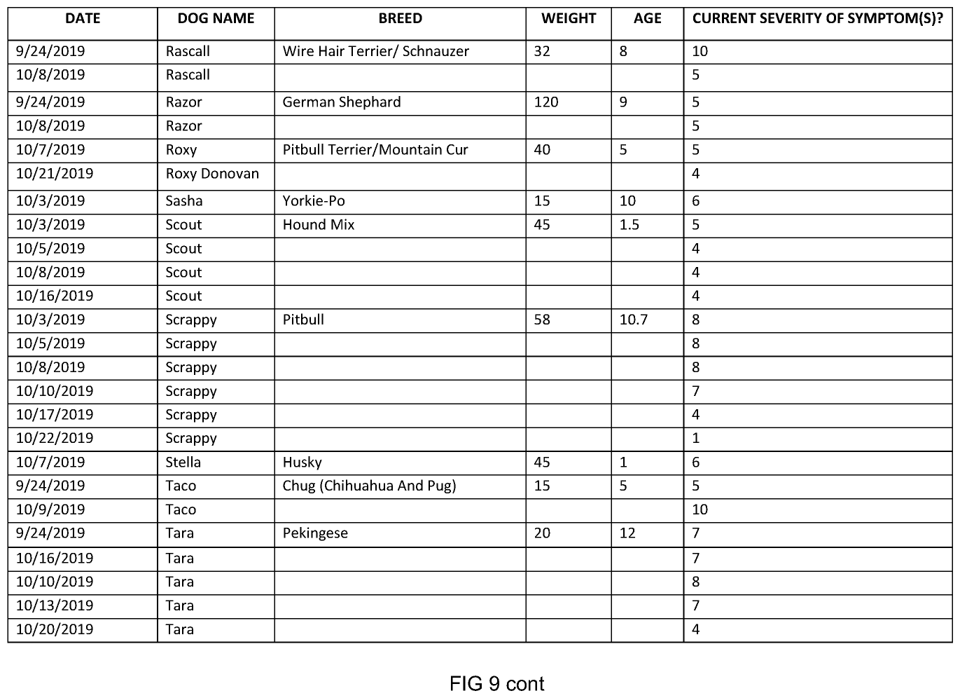

The following study data appendix includes data tables 1-7 (shown in FIG. 3-FIG. 9) which provide information about the cohort of dogs enrolled in the study, including breed, age, weight, health and behaviour issues and observations. (Severity Score means: 1=NONE, 10=EXTREMELY SEVERE).

What is claimed is:

1. A dietary supplement composition comprising:
one or more cannabinoids;
one or more organosulfur compounds;
one or more medium-chain triglycerides;
colloidal silver; and
an edible carrier.

2. The dietary supplement composition of claim 1, further comprising ginger and optionally comprising one or more flavorings, proteins selected from animal and vegetable derived proteins, and any one or more fruits, vegetables and meats and combinations of these.

3. The dietary supplement composition of claim 2 wherein the composition demonstrates a shelf life characterized as having a shelf life increase up to an additional 5-6 weeks without refrigeration as compared with compositions lacking at least one or both MCT and ginger.

4. The dietary supplement composition of claim 2, wherein the composition is provided in an edible biscuit form that is prepared from a mixture of both dry and liquid ingredients with the CBD and MSM combination

5. The dietary supplement composition of claim 4, wherein the composition is prepared from a mixture comprising dry ingredients comprising CBD, MSM, whole meal flour, baking powder, and ginger, and liquid ingredients comprising Silver Hydrosol, MCT palm oil, milk, and peanut butter.

6. The dietary supplement composition of claim 1, wherein the one or more cannabinoids comprises cannabidiol and the one or more organosulfur compounds comprises methyl sulfonylmethane.

7. The dietary supplement composition of claim 1, further comprising ginger and optionally comprising one or more flavorings, proteins selected from animal and vegetable derived proteins, and any one or more fruits, vegetables and meats and combinations of these.

8. A method of administering a cannabinoid to a canine comprising:
orally administering a cannabinoid composition to a canine;
wherein the cannabinoid composition comprises,
one or more cannabinoids;
one or more organosulfur compounds;
one or more medium-chain triglycerides;
colloidal silver; and
an edible carrier.

9. The method of claim 8, wherein the one or more cannabinoids includes cannabidiol (CBD).

10. The method of claim 9, wherein the CBD is present in an amount of about 0.5 weight percent to about 3 weight percent, based on the total weight of the cannabinoid composition.

11. The method of claim 8, wherein the one or more organosulfur compounds includes methylsulfonylmethane (MSM).

12. The method of claim 8, wherein the edible carrier includes ginger.

13. The method of claim 8, wherein the amount of cannabidiol administered to the canine in a 24-hour period is between 0.134 milligrams per kilogram of the canine and 0.157 milligrams per kilogram of the canine.
